(19) United States
(12) Patent Application Publication
(10) Pub. No.: US 2007/0093777 A1
(43) Pub. Date: Apr. 26, 2007

(54) Water therapy apparatus for large intestine

(76) Inventor: Hsin-Ming Chen, Taipei (TW)

Correspondence Address: COOPER & DUNHAM, LLP, 1185 AVENUE OF THE AMERICAS, NEW YORK, NY, 10036, US

(21) Appl. No.: 11/257,690

(22) Filed: Oct. 25, 2005

(51) Int. Cl. A61M 31/00

(52) U.S. Cl. 604/500

(57) ABSTRACT

A water therapy apparatus for large intestine has a rib mounted under a rear rim of a toilet seat and a controller mounted on an outer wall of a toilet bowl. The rib has a guide tube to connect to a nozzle, which is inserted into the large intestine of a user. The controller has an inlet tube, which is connected to a faucet and an outlet tube, which is connected to the guide tube. Hence, water flows from the faucet to the controller, filtered, heated and pressurized, through the outlet tube, the guide tube and sprayed from the nozzle to enter the large intestine of the user. Furthermore, a spray tube is mounted adjacent to the rib and connected to an outlet tube so that the water can be sprayed from the spray tube to clean an outer surface of the anus of the user.

### BACKGROUND OF THE INVENTION

1. Field of the Invention

The present invention relates to a water therapy apparatus for the large intestine, and more particularly to a water therapy apparatus that is easy to use in the home.

2. Description of the Related Art

In water therapy for the large intestine, also known as an enema, warm water is guided into the large intestine of a person to help soften stool in the bowels so that it is easy for the person to pass the stool.

A conventional water therapy apparatus, however, requires a large volume of water and it is inconvenient for a person to use the conventional water therapy apparatus at home for self-administered therapy. Furthermore, it is difficult for a person to purchase one due to high cost.

In addition, a conventional toilet may have a built-in bidet, a spray tube mounted under a rear end of a toilet seat. The spray tube aims at the anus of a user when the user is sitting on the seat and cleans the outer surface of the anus with water sprayed from the spray tube. This apparatus is useful for a person suffering from hemorrhoids. However, the spray tube of a conventional toilet with a built-in bidet cannot guide water into the large intestine of a person to help soften stool.

Therefore, the invention provides a water therapy apparatus for large intestine to mitigate or obviate the aforementioned problems.

### SUMMARY OF THE INVENTION

The main objective of the present invention is to provide a water therapy apparatus for the large intestine that is easy to use in the home.

Other objectives, advantages and novel features of the invention will become more apparent from the following detailed description when taken in conjunction with the accompanying drawings.

### BRIEF DESCRIPTION OF THE DRAWINGS

FIG. 1 is a perspective view of a first preferred embodiment of a water therapy apparatus for the large intestine in accordance with the present invention;

FIG. 2 is a block flow diagram of devices in a controller of the first preferred embodiment of the water therapy apparatus for the large intestine in FIG. 1;

FIG. 3 is a perspective view of a second embodiment of a water therapy apparatus for the large intestine in accordance with the present invention;

FIG. 4 is a perspective view of a third embodiment of a water therapy apparatus for the large intestine in accordance with the present invention;

FIG. 5 is a perspective view of a fourth embodiment of a water therapy apparatus for the large intestine in accordance with the present invention; and

FIG. 6 is an operational perspective view of the water therapy apparatus for the large intestine in accordance with the present invention.

### DETAILED DESCRIPTION OF THE PREFERRED EMBODIMENT

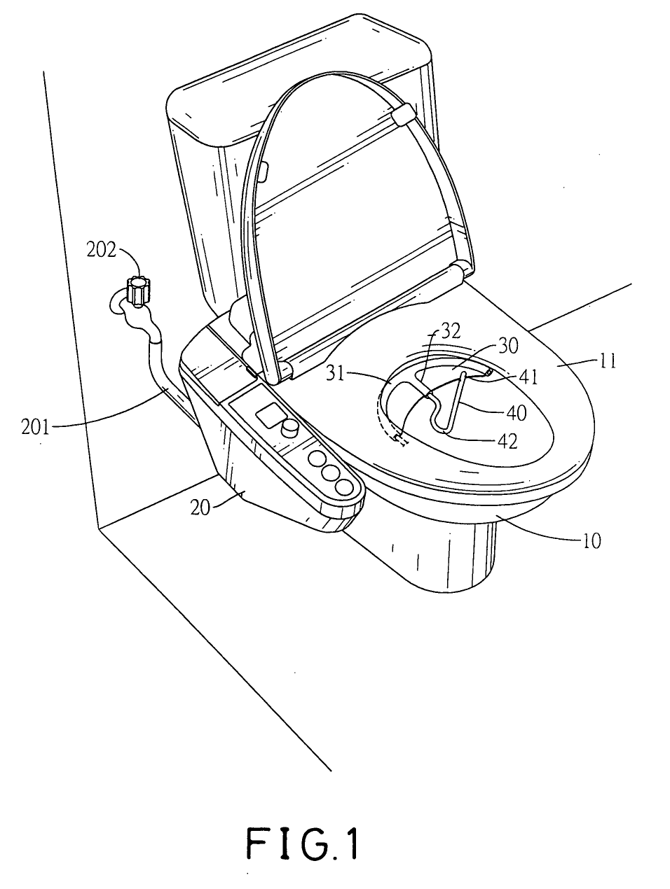

With reference to FIG. 1, a first preferred embodiment of a water therapy apparatus for the large intestine comprises a toilet seat (11), a controller (20) and a rib (30). The toilet seat (11) is attached to a toilet bowl (10) and has a rear rim. The controller (20) is mounted in an outer rim of a toilet bowl and the rib (30) is mounted under a rear edge of the toilet seat (11).

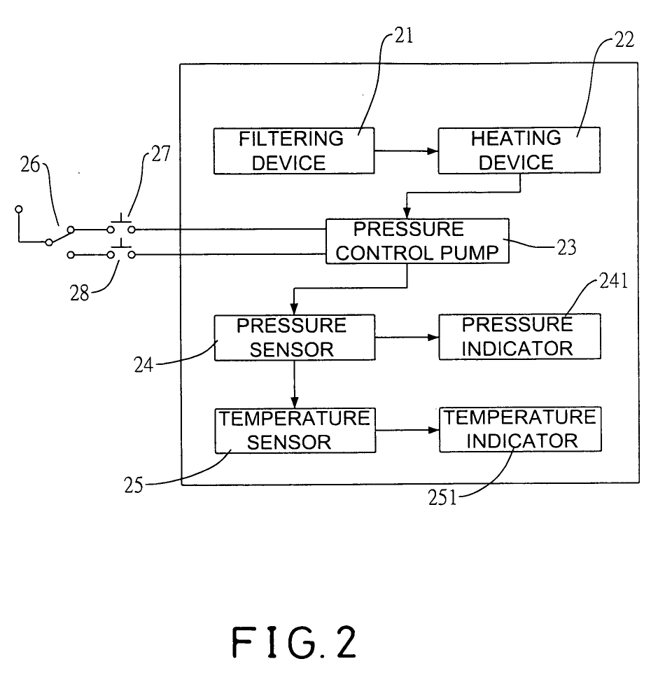

With further reference to FIG. 2, the controller has a filtering device (21), a heating device (22), a pressure control pump (23), a pressure sensor (24), a temperature sensor (25), a pressure indicator (241) and a temperature indicator (251). The pressure control pump (23) is connected to a switch device, which comprises a selector switch (26), a pressure switch (27) and a toggle switch (28). The pressure switch (27) allows for intermittent operation of the water therapy apparatus. When the pressure switch (27) is pressed and held, the controller (20) operates. Alternatively, when the pressure switch (27) is released, the controller (20) stops at once. The toggle switch (28) allows for continuous operation. When the toggle switch (28) is pressed once the controller (20) is turned on and operates. When the toggle switch (28) is pressed a second time the controller (20) is tuned off and stops. The selector switch (26) allows a user to choose between intermittent or continuous operation of the water therapy apparatus.

An inlet tube (201) is provided on a rear end of the controller (20) and an outlet tube is also connected to the controller (20). The filtering device (21) and heating device (22) are both provided on the inlet tube (201) while the pressure sensor (24) and the temperature sensor (25) are both provided on the outlet tube.

The pressure sensor (24) is connected to the pressure control pump (23) to detect the water pressure, and the pressure indicator (241) is connected to the pressure sensor (24) to show off the detected pressure. The temperature sensor (25) is connected to the pressure sensor (24) to detect the water temperature, and the temperature indicator (251) is connected to the temperature sensor (25) to show off the detected water temperature.

The rib (30) has a guide tube (31) formed on an end thereof at the toilet seat and a pipe (32) is integrally formed in a middle portion of the guide tube (31) and extends forward. A nozzle (40), which is a soft tube, has multiple spray holes (41) defined in a top end thereof and a trap (42) formed in a bottom end thereof. The nozzle (40) is mounted upright in a free end of the pipe (32).

A first end of the inlet tube (201) is connected to the pressure control pump (23) and a second end of the inlet tube (201) is connected to a faucet (202). In the first preferred embodiment of the present invention, the faucet (202) is mounted on a wall in a bathroom. A first end of the outlet tube is connected to the pressure control pump (23) and a second end of the outlet tube is connected to the guide tube (31).

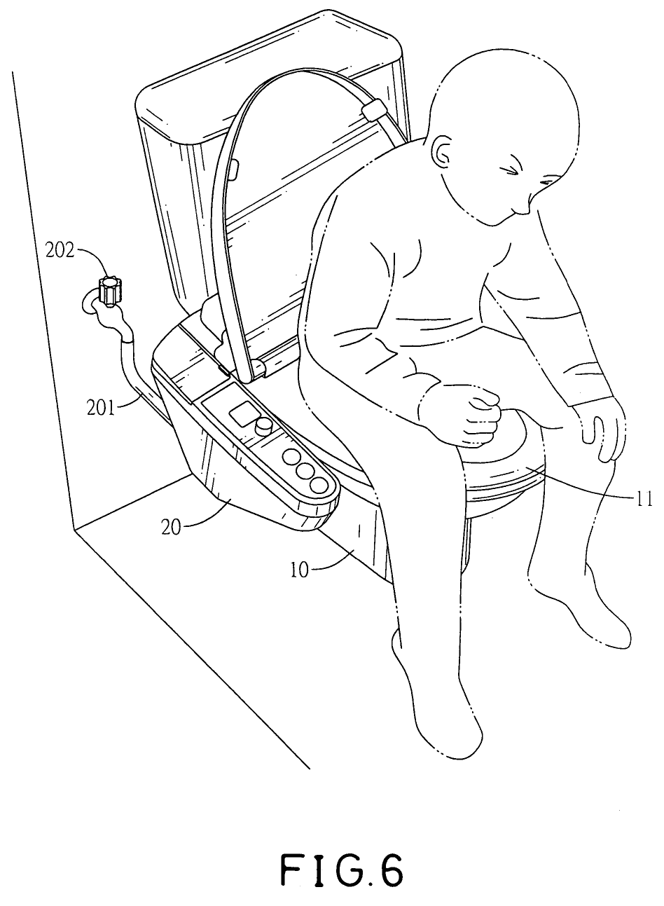

With further reference to FIG. 6, when a user is sitting on the toilet seat (11) with the present invention, the nozzle (40) is inserted into rectum of the user for therapy. Because of the trap (42), it is easy for the user to control the insertion of the nozzle (40) and feel comfortable. The user can choose the intermittent or the continuous method of the therapy. During the therapy, the user can view the pressure indicator (241) and the temperature indicator (251) to monitor the operating conditions of the controller (20).

When the controller (20) is operating, water supplied from the faucet (202) flows through the inlet tube (201) to the pressure control pump (23). The water in the inlet tube (201) is cleaned by the filtering device (21) and adjusted to a desired temperature by the heating device (22). The water, the pressure of which is controlled by the pressure control device (23), is then transferred from the outlet tube and into the guide tube (31). Finally, the water flows into the pipe (32) and the nozzle (40) in turn, then sprayed from the spray holes (41). When the therapy is finished, the nozzle (40) can be detached from the controller (20) for renewal.

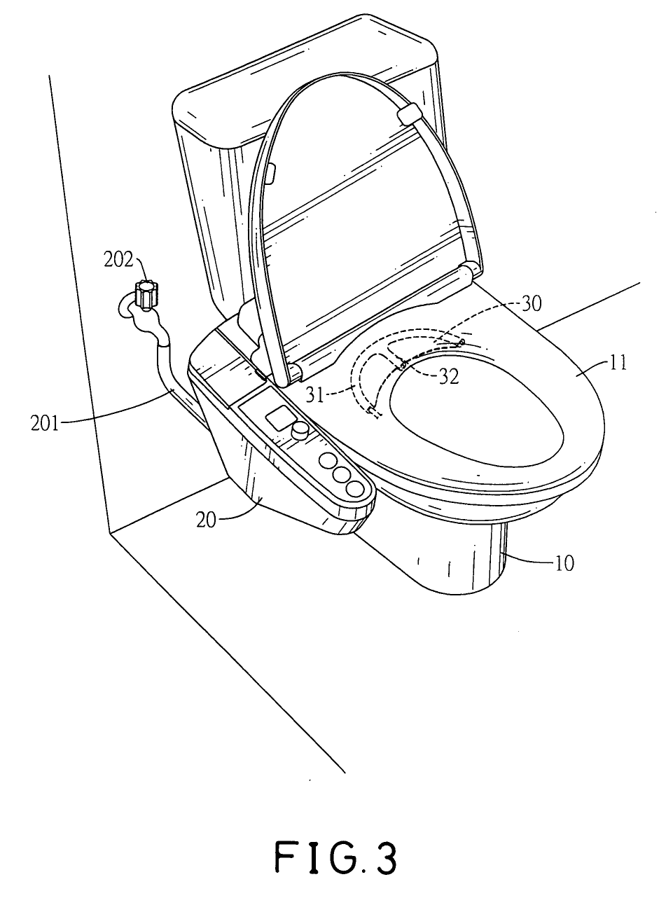

With reference to FIG. 3, a second embodiment of a water therapy apparatus for the large intestine is similar to the first preferred embodiment except that a space is defined between the toilet seat (11) and the toilet bowl (10) so that the rib (30) can be moved rearward and be received in the space for storage and to prevent soiling when not in use. For use, the rib (30) is pulled forward to a desired position at the anus of the user and inserted into the large intestine.

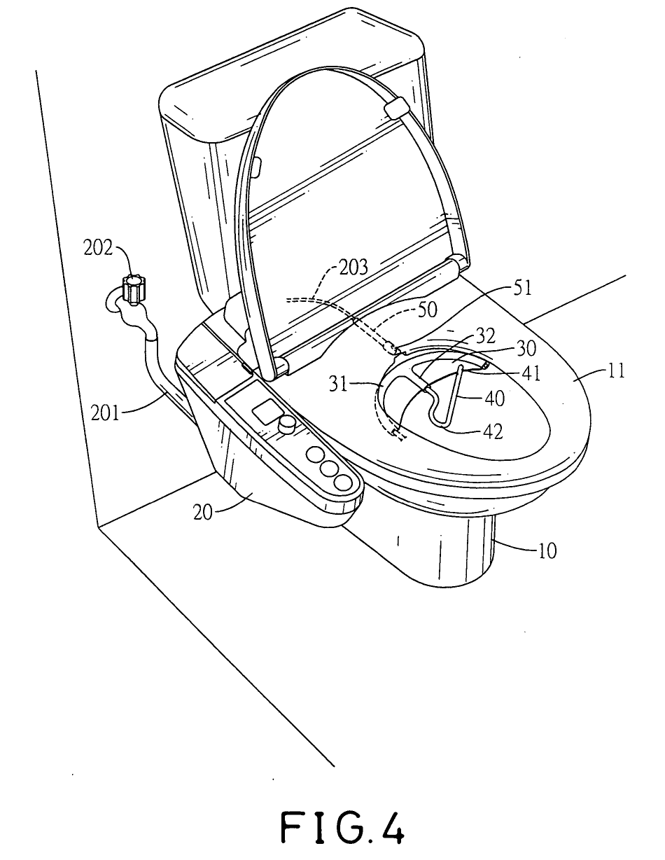

With reference to FIG. 4, a third embodiment of a therapy apparatus for the large intestine is similar to the second embodiment except that a spray tube (50) is mounted under the rear rim of the seat (11) and behind the rib (30) to clean the anus of the user. The spray tube (50) has multiple holes (51) defined in an end thereof. A first end of the outlet tube (203) is connected to the spray tube (50) and a second end is connected to the pressure control pump (23). In the process of cleaning the anus, the rib (30) is detached from the toilet and the spray tube (50) is moved forward or rearward by the controller (20) to accurately aim the spray tube (50) at the anus of the user. Warm filtered water at a desired pressure flows through the outlet tube (203) and is sprayed from the holes (51) of the spray tube (50) to clean the outer surface of the anus.

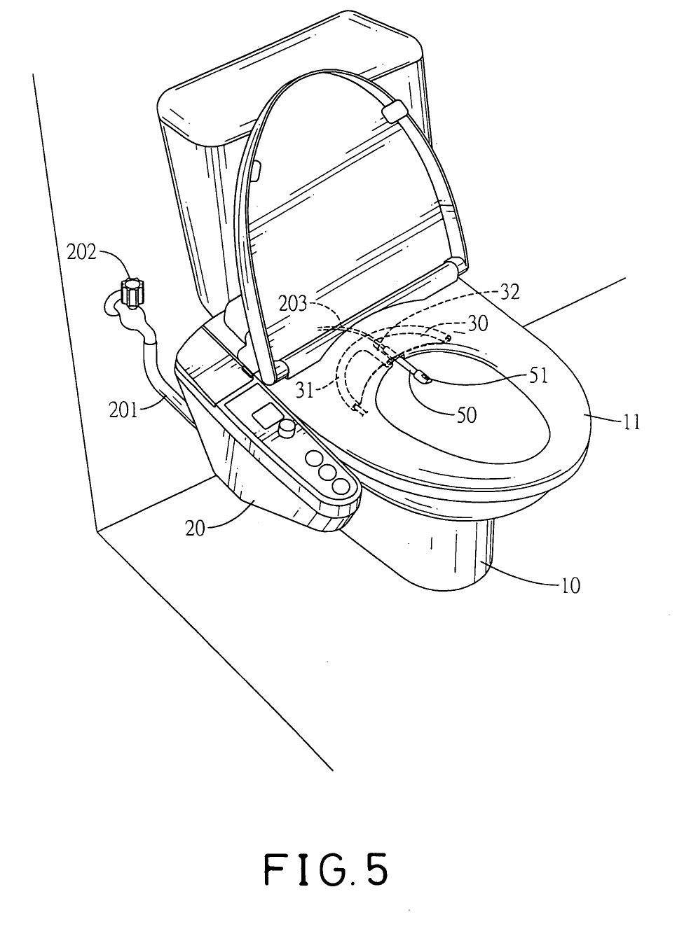

With reference to FIG. 5, a fourth embodiment of a therapy apparatus for the large intestine is similar to the third embodiment except that a space is defined between the toilet seat (11) and the toilet bowl (10) so that the rib (30) can be moved rearward and be received in the space for storage and to prevent soiling. For use, the rib (30) is pulled forward to a desired position to at the anus of the user and the nozzle (40) inserted into the large intestine.

Hence, the preferred embodiment of the present invention can be mounted on a toilet in a home conveniently and economically. Furthermore, to meet the requirements of the user, the present invention can be used to clean the outer surface of the anus and the large intestine alternatively.

It is to be understood, however, that even though numerous characteristics and advantages of the present invention have been set forth in the 1 foregoing description, together with details of the structure and function of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. A water therapy apparatus for the large intestine comprising:
a toilet seat adapted to be mounted on a toilet bowl and having a rear end and a rear rim,
a rib mounted under the rear rim of the toilet seat and having
a guide tube formed on an end of the rib; and
a controller adapted for being mounted on an outer wall of the toilet bowl, connected to the rib and having
a switch device,
a pressure control pump connected to the switch device,
a heating device connected to the pressure control pump,
a filtering device connected to the heating device,
a nozzle connected to the rib,
an inlet tube, a first end of the inlet tube connected to the pressure control pump and a second end of the inlet tube adapted to be connected to a faucet, and
an outlet tube, a first end of the outlet tube connected to the pressure control pump and a second end of the outlet tube connected to the guide tube.

2. The water therapy apparatus for the large intestine as claimed in claim 1, wherein the rib is securely provided under the rear rim of the toilet seat.

3. The water therapy apparatus for the large intestine as claimed in claim 1, wherein the rib is moved rearward and received between the rear end of the toilet seat for storage and the rib is moved forward to a position for use.

4. The water therapy apparatus for the large intestine as claimed in claim 1, wherein the controller has a pressure sensor connected to the pressure control pump, a temperature sensor connected to the pressure sensor, a pressure indicator connected to the pressure sensor and a temperature indicator connected to the temperature sensor, wherein the pressure sensor and the temperature sensor are both connected to the outer tube.

5. The water therapy apparatus for the large intestine as claimed in claim 1, wherein a pipe is formed on a middle portion of the guide tube and sleeved around the nozzle, the nozzle is a soft tube and multiple spray holes are respectively defined in a top end of the nozzle.

6. The water therapy apparatus for the large intestine as claimed in claim 5, wherein a trap is formed in a bottom end of the nozzle.

7. A water therapy apparatus for the large intestine comprising:
a toilet seat adapted to be mounted on a toilet bowl and having a rear end and a rear rim,
a rib mounted under the rear rim of the toilet seat and having
a guide tube formed on an end of the rib at the toilet seat;
a controller adapted to be mounted on an outer wall of a toilet bowl, connected to the rib and having
a switch device,
a pressure control pump connected to the switch device,
a heating device connected to the pressure control pump,
a filtering device connected to the heating device,
a nozzle connected to the rib, and
an inlet tube, a first end of the inlet tube connected to the pressure control pump and a second end of the inlet tube adapted to be connected to a faucet,
a spray tube mounted under the rear rim of the toilet seat and behind the rib, controlled by the controller to move forward or rearward and having
multiple holes defined in an end thereof, and
an outlet tube, a first end of which is connected to the spray tube and a second end of which is connected to the pressure control pump.

8. The water therapy apparatus for the large intestine as claimed in claim 7, wherein the rib is securely provided under the rear rim of the toilet seat.

9. The water therapy apparatus for the large intestine as claimed in claim 7, wherein the rib is moved rearward and received between the rear end of the toilet seat for storage and the rib is moved forward to a position for use.

10. The water therapy apparatus for the large intestine as claimed in claim 7, wherein the controller has a pressure sensor connected to the pressure control pump, a temperature sensor connected to the pressure sensor, a pressure indicator connected to the pressure sensor and a temperature indicator connected to the temperature sensor, wherein the pressure sensor and the temperature sensor are both connected to the outer tube.

11. The water therapy apparatus for the large intestine as claimed in claim 7, wherein a pipe is formed on a middle portion of the guide tube and sleeved around the upright nozzle, the nozzle is a soft tube and multiple spray holes are respectively defined in a top end of the nozzle.

12. The water therapy apparatus for the large intestine as claimed in claim 11, wherein a trap is formed in a bottom end of the nozzle.
